SEQUENCE LISTING (32 sequences)

SEQ ID NO: 1 - DNA, 9420 bp, Homo sapiens
cagcgactcctctggctcccgagaagtggatccggtcgcggccactacgatgccgggagccgccggggtcctcctccttctgctgctctccggaggcctcgggggcgtacaggcgcagcggccgcagcagcagcggcagtcacaggcacatcagcaaagaggtttattccctgctgtcctgaatcttgcttctaatgctcttatcacgaccaatgcaacatgtggagaaaaaggacctgaaatgtactgcaaattggtagaacatgtccctgggcagcctgtgaggaacccgcagtgtcgaatctgcaatcaaaacagcagcaatccaaaccagagacacccgattacaaatgctattgatggaaagaacacttggtggcagagtcccagtattaagaatggaatcgaataccattatgtgacaattacactggatttacagcaggtgttccagatcgcgtatgtgattgtgaaggcagctaactccccccggcctggaaactggattttggaacgctctcttgatgatgttgaatacaagccctggcagtatcatgctgtgacagacacggagtgcctaacgctttacaatatttatccccgcactgggccaccgtcatatgccaaagatgatgaggtcatctgcacttcattttactccaagatacaccccttagaaaatggagagattcacatctctttaatcaatgggagaccaagtgccgatgatccttctccagaactgctagaatttacctccgctcgctatattcgcctgagatttcagaggatccgcacactgaatgctgacttgatgatgtttgctcacaaagacccaagagaaattgaccccattgtcaccagaagatattactactcggtcaaggatatttcagttggagggatgtgcatctgctatggtcatgccagggcttgtccacttgatccagcgacaaataaatctcgctgtgagtgtgagcataacacatgtggcgatagctgtgatcagtgctgtccaggattccatcagaaaccctggagagctggaacttttctaactaaaactgaatgtgaagcatgcaattgtcatggaaaagctgaagaatgctattatgatgaaaatgttgccagaagaaatctgagtttgaatatacgtggaaagtacattggagggggtgtctgcattaattgtacccaaaacactgctggtataaactgcgagacatgtacagatggcttcttcagacccaaaggggtatctccaaattatccaaggccatgccagccatgtcattgcgatccaattggttccttaaatgaagtctgtgtcaaggatgagaaacatgctcgacgaggtttggcacctggatcctgtcattgcaaaactggttttggaggtgtgagctgtgatcggtgtgccaggggctacactggctacccggactgcaaagcctgtaactgcagtgggttagggagcaaaaatgaggatccttgttttggcccctgtatctgcaaggaaaatgttgaaggaggagactgtagtcgttgcaaatccggcttcttcaatttgcaagaggataattggaaaggctgcgatgagtgtttctgttcaggggtttcaaacagatgtcagagttcctactggacctatggcaaaatacaagatatgagtggctggtatctgactgaccttcctggccgcattcgagtggctccccagcaggacgacttggactcacctcagcagatcagcatcagtaacgcggaggcccggcaagccctgccgcacagctactactggagcgcgccggctccctatctgggaaacaaactcccagcagtaggaggacagttgacatttaccatatcatatgaccttgaagaagaggaagaagatacagaacgtgttctccagcttatgattatcttagagggtaatgacttgagcatcagcacagcccaagatgaggtgtacctgcacccatctgaagaacatactaatgtattgttacttaaagaagaatcatttaccatacatggcacacattttccagtccgtagaaaggaatttatgacagtgcttgcgaatttgaagagagtcctcctacaaatcacatacagctttgggatggatgccatcttcaggttgagctctgttaaccttgaatccgctgtctcctatcctactgatggaagcattgcagcagctgtagaagtgtgtcagtgcccaccagggtatactggctcctcttgtgaatcttgttggcctaggcacaggcgagttaacggcactatttttggtggcatctgtgagccatgtcagtgctttggtcatgcggagtcctgtgatgacgtcactggagaatgcctgaactgtaaggatcacacaggtggcccatattgtgataaatgtcttcctggtttctatggcgagcctactaaaggaacctctgaagactgtcaaccctgtgcctgtccactcaatatcccatccaataactttagcccaacgtgccatttagaccggagtcttggattgatctgtgatggatgccctgtcgggtacacaggaccacgctgtgagaggtgtgcagaaggctattttggacaaccctctgtacctggaggatcatgtcagccatgccaatgcaatgacaaccttgacttctccatccctggcagctgtgacagcttgtctggctcctgtctgatatgtaaaccaggtacaacaggccggtactgtgagctctgtgctgatggatattttggagatgcagttgatgcgaagaactgtcagccctgtcgctgtaatgccggtggctctttctctgaggtttgccacagtcaaactggacagtgtgagtgcagagccaacgttcagggtcagagatgtgacaaatgcaaggctgggacctttggcctacaatcagcaaggggctgtgttccctgcaactgcaattcttttgggtctaagtcattcgactgtgaagagagtggacaatgttggtgccaacctggagtcacagggaagaaatgtgaccgctgtgcccacggctatttcaacttccaagaaggaggctgcacagcttgtgaatgttctcatctgggtaataattgtgacccaaagactgggcgatgcatttgcccacccaataccattggagagaaatgttctaaatgtgcacccaatacctggggccacagcattaccactggttgtaaggcttgtaactgcagcacagtgggatccttggatttccaatgcaatgtaaatacaggccaatgcaactgtcatccaaaattctctggtgcaaaatgtacagagtgcagtcgaggtcactggaactaccctcgctgcaatctctgtgactgcttcctccctgggacagatgccacaacctgtgattcagagactaaaaaatgctcctgtagtgatcaaactgggcagtgcacttgtaaggtgaatgtggaaggcatccactgtgacagatgccggcctggcaaattcggactcgatgccaagaatccacttggctgcagcagctgctattgcttcggcactactacccagtgctctgaagcaaaaggactgatccggacgtgggtgactctgaaggctgagcagaccattctacccctggtagatgaggctctgcagcacacgaccaccaagggcattgtttttcaacatccagagattgttgcccacatggacctgatgagagaagatctccatttggaacctttttattggaaacttccagaacaatttgaaggaaagaagttgatggcctatgggggcaaactcaagtatgcaatctatttcgaggctcgggaagaaacaggtttctctacatataatcctcaagtgatcattcgaggtgggacacctactcatgctagaattatcgtcaggcatatggctgctcctctgattggccaattgacaaggcatgaaattgaaatgacagagaaagaatggaaatattatggggatgatcctcgagtccatagaactgtgacccgagaagacttcttggatatactatatgatattcattacattcttatcaaagctacttatggaaatttcatgcgacaaagcaggatttctgaaatctcaatggaggtagctgaacaaggacgtggaacaacaatgactcctccagctgacttgattgaaaaatgtgattgtcccctgggctattctggcctgtcctgtgaggcatgcttgccgggattttatcgactgcgttctcaaccaggtggccgcacccctggaccaaccctgggcacctgtgttccatgtcaatgtaatggacacagcagcctgtgtgaccctgaaacatcgatatgccagaattgtcaacatcacactgctggtgacttctgtgaacgatgtgctcttggatactatggaattgtcaagggattgccaaatgactgtcagcaatgtgcctgccctctgatttcttccagtaacaatttcagcccctcttgtgtcgcagaaggacttgacgactaccgctgcacggcttgtccacggggatatgaaggccagtactgtgaaaggtgtgcccctggctatactggcagtccaggcaaccctggaggctcctgccaagaatgtgagtgtgatccctatggctcactgcctgtgccctgtgaccctgtcacaggattctgcacgtgccgacctggagccacgggaaggaagtgtgacggctgcaagcactggcatgcacgcgagggctgggagtgtgttttttgtggagatgagtgcactggccttcttctcggtgacttggctcgcctggagcagatggtcatgagcatcaacctcactggtccgctgcctgcgccatataaaatgctgtatggtcttgaaaatatgactcaggagctaaagcacttgctgtcacctcagcgggccccagagaggcttattcagctggcagagggcaatctgaatacactcgtgaccgaaatgaacgagctgctgaccagggctaccaaagtgacagcagatggcgagcagaccggacaggatgctgagaggaccaacacaagagcaaagtccctgggagaattcattaaggagcttgcccgggatgcagaagctgtaaatgaaaaagctataaaactaaatgaaactctaggaactcgagacgaggcctttgagagaaatttggaagggcttcagaaagagattgaccagatgattaaagaactgaggaggaaaaatctagagacacaaaaggaaattgctgaagatgagttggtagctgcagaagcccttctgaaaaaagtgaagaagctgtttggagagtcccggggggaaaatgaagaaatggagaaggatctccgggaaaaactggctgactacaaaaacaaagttgatgatgcttgggaccttttgagagaagccacagataaaatcagagaagctaatcgcctatttgcagtaaatcagaaaaacatgactgcattggagaaaaagaaggaggctgttgagagcggcaaacgacaaattgagaacactttaaaagaaggcaatgacatactcgatgaagccaaccgtcttgcagatgaaatcaactccatcatagactatgttgaagacatccaaactaaattgccacctatgtctgaggagcttaatgataaaatagatgacctctcccaagaaataaaggacaggaagcttgctgagaaggtgtcccaggctgagagccacgcagctcagttgaatgactcatctgctgtccttgatggaatccttgatgaggctaaaaacatctccttcaatgccactgcagccttcaaagcttacagcaatattaaggactatattgatgaagctgagaaagttgccaaagaagccaaagatcttgcacatgaagctacaaaactggcaacaggtcctcggggtttattaaaggaagatgccaaaggctgtcttcagaaaagcttcaggattcttaacgaagccaagaagttagcaaatgatgtaaaagaaaatgaagaccatctaaatggcttaaaaaccaggatagaaaatgctgatgctagaaatggggatctcttgagaactttgaatgacactttgggaaagttatcagctattccaaatgatacagctgctaaactgcaagctgttaaggacaaagccagacaagccaacgacacagctaaagatgtactggcacagattacagagctccaccagaacctcgatggcctgaagaagaattacaataaactagcagacagcgtcgccaaaacgaatgctgtggttaaagatccttccaagaacaaaatcattgccgatgcagatgccactgtcaaaaatttagaacaggaagctgaccggctaatagataaactcaaacccatcaaggaacttgaggataacctaaagaaaaacatctctgagataaaggaattgataaaccaagctcggaaacaagccaattctatcaaagtatctgtgtcttcaggaggtgactgcattcgaacatacaaaccagaaatcaagaaaggaagttacaataatattgttgtcaacgtaaagacagctgttgctgataacctcctcttttatcttggaagtgccaaatttattgactttctggctatagaaatgcgtaaaggcaaagtcagcttcctctgggatgttggatctggagttggacgtgtagagtacccagatttgactattgatgactcatattggtaccgtatcgtagcatcaagaactgggagaaatggaactatttctgtgagagccctggatggacccaaagccagcattgtgcccagcacacaccattcgacgtctcctccagggtacacgattctagatgtggatgcaaatgcaatgctgtttgttggtggcctgactgggaaattaaagaaggctgatgctgtacgtgtgattacattcactggctgcatgggagaaacatactttgacaacaaacctataggtttgtggaatttccgagaaaaagaaggtgactgcaaaggatgcactgtcagtcctcaggtggaagatagtgaggggactattcaatttgatggagaaggttatgcattggtcagccgtcccattcgctggtaccccaacatctccactgtcatgttcaagttcagaacattttcttcgagtgctcttctgatgtatcttgccacacgagacctgagagatttcatgagtgtggagctcactgatgggcacataaaagtcagttacgatctgggctcaggaatggcttccgttgtcagcaatcaaaaccataatgatgggaaatggaaatcattcactctgtcaagaattcaaaaacaagccaatatatcaattgtagatatagatactaatcaggaggagaatatagcaacttcgtcttctggaaacaactttggtcttgacttgaaagcagatgacaaaatatattttggtggcctgccaacgctgagaaacttgagtatgaaagcaaggccagaagtaaatctgaagaaatattccggctgcctcaaagatattgaaatttcaagaactccgtacaatatactcagtagtcccgattatgttggtgttaccaaaggatgttccctggagaatgtttacacagttagctttcctaagcctggttttgtggagctctcccctgtgccaattgatgtaggaacagaaatcaacctgtcattcagcaccaagaatgagtccggcatcattcttttgggaagtggagggacaccagcaccacctaggagaaaacgaaggcagactggacaggcctattatgtaatactcctcaacaggggccgtctggaagtgcatctctccacaggggcacgaacaatgaggaaaattgtcatcagaccagagccgaatctgtttcatgatggaagagaacattccgttcatgtagagcgaactagaggcatctttacagttcaagtggatgaaaacagaagatacatgcaaaacctgacagttgaacagcctatcgaagttaaaaagcttttcgttgggggtgctccacctgaatttcaaccttccccactcagaaatattcctccttttgaaggctgcatatggaatcttgttattaactctgtccccatggactttgcaaggcctgtgtccttcaaaaatgctgacattggtcgctgtgcccatcagaaactccgtgaagatgaagatggagcagctccagctgaaatagttatccagcctgagccagttcccaccccagcctttcctacgcccaccccagttctgacacatggtccttgtgctgcagaatcagaaccagctcttttgatagggagcaagcagttcgggctttcaagaaacagtcacattgcaattgcatttgatgacaccaaagttaaaaaccgtctcacaattgagttggaagtaagaaccgaagctgaatccggcttgcttttttacatggctcgcatcaatcatgctgattttgcaacagttcagctgagaaatggattgccctacttcagctatgacttggggagtggggacacccacaccatgatccccaccaaaatcaatgatggccagtggcacaagattaagataatgagaagtaagcaagaaggaattctttatgtagatggggcttccaacagaaccatcagtcccaaaaaagccgacatcctggatgtcgtgggaatgctgtatgttggtgggttacccatcaactacactacccgaagaattggtccagtgacctatagcattgatggctgcgtcaggaatctccacatggcagaggcccctgccgatctggaacaacccacctccagcttccatgttgggacatgttttgcaaatgctcagaggggaacatattttgacggaaccggttttgccaaagcagttggtggattcaaagtgggattggaccttcttgtagaatttgaattccgcacaactacaacgactggagttcttctggggatcagtagtcaaaaaatggatggaatgggtattgaaatgattgatgaaaagttgatgtttcatgtggacaatggtgcgggcagattcactgctgtctatgatgctggggttccagggcatttgtgtgatggacaatggcataaagtcactgccaacaagatcaaacaccgcattgagctcacagtcgatgggaaccaggtggaagcccaaagcccaaacccagcatctacatcagctgacacaaatgaccctgtgtttgttggaggcttcccagatgacctcaagcagtttggcctaacaaccagtattccgttccgaggttgcatcagatccctgaagctcaccaaaggcacaggcaagccactggaggttaattttgccaaggccctggaactgaggggcgttcaacctgtatcatgcccagccaactaata

SEQ ID NO: 2 - DNA, 619 bp, Homo sapiens
tgcagcttccttctcaccttgaagaataatcctagaaaactcacaaaatgtgtgatgcttttgtaggtacctggaaacttgtctccagtgaaaactttgatgattatatgaaagaagtaggagtgggctttgccaccaggaaagtggctggcatggccaaacctaacatgatcatcagtgtgaatggggatgtgatcaccattaaatctgaaagtacctttaaaaatactgagatttccttcatactgggccaggaatttgacgaagtcactgcagatgacaggaaagtcaagagcaccataaccttagatgggggtgtcctggtacatgtgcagaaatgggatggaaaatcaaccaccataaagagaaaacgagaggatgataaactggtggtggaatgcgtcatgaaaggcgtcacttccacgagagtttatgagagagcataagccaagggacgttgacctggactgaagttcgcattgaactctacaacattctgtgggatatattgttcaaaaagatattgttgttttccctgatttagcaagcaagtaattttctcccaagctgattttattcaatatggttacgttggttaaataactttttttagatttag

SEQ ID NO: 3 - DNA, 976 bp, Homo sapiens
tattgagaggaacaaagagcttataaatacattaggacctggaattcggttgtccagccacaaaggtgacagcatttaacaaagcttagagaaacctccaggagactgctaccatggcagagaagcccaagctccactactccaatatacggggcagaatggagtccatccggtggctcctggctgcagctggagtagagtttgaagagaaatttataaaatctgcagaagatttggacaagttaagaaatgatggatatttgatgttccagcaagtgccaatggttgagattgatgggatgaagctggtgcagaccagagccattctcaactacattgccagcaaatacaacctctatgggaaagacataaaggagaaagccctgattgatatgtatatagaaggtatagcagatttgggtgaaatgatccttcttctgccctttactcaacctgaggaacaagatgccaagcttgccttgatccaagagaaaacaaaaaatcgctacttccctgcctttgaaaaagtcttaaagagccacggacaagactaccttgttggcaacaagctgagccgggctgacattcacctggtggaacttctctactacgtggaagagcttgactctagccttatttccagcttccctctgctgaaggccctgaaaaccagaatcagtaacctgcccacagtgaagaagtttctacagcctggcagcccaaggaagcctcccatggatgagaaatctttagaagaatcaaggaagattttcaggttttaataaaccagccatagaggtcaagaacatgcaagaccagtattctaaagttttgcaacaattaagtgctttacctaagtgttgattgtgcctgttgtgaagctaatgaactctttcaaattatatgctaattaaataatacaactcctattcacccacttagttaaaattgatttcttctcattaggatctgatgt

SEQ ID NO: 4 - DNA, 696 bp, Homo sapiens
ccactgctcaggtctgcacatccctacaatggctaaaacagcaatggcctacaaggaaaaaatgaaggagctgtccatgctgtcactgatctgctcttgcttttacccggaacctcgcaacatcaacatctatacttacgatgatatggaagtgaagcaaatcaacaaacgtgcctctggccaggcttttgagctgatcttgaagccaccatctcctatctcagaagccccacgaactttagcttctccaaagaagaaagacctgtccctggaggagatccagaagaaactggaggctgcaggggaaagaagaaagtctcaggaggcccaggtgctgaaacaattggcagagaagagggaacacgagcgagaagtccttcagaaggctttggaggagaacaacaacttcagcaagatggcggaggaaaagctgatcctgaaaatggaacaaattaaggaaaaccgtgaggctaatctagctgctattattgaacgtctgcaggaaaaggagaggcatgctgcggaggtgcgcaggaacaaggaactccaggttgaactgtctggctgaagcaagggagggtctggcacgccccaccaatagtaaatccccctgcctatatttataatggatcatgcgatatcatgcgatatcaggatggggaattgatgacaatggtttaaaagactctattataa

SEQ ID NO: 5 - DNA, 2790 bp, Homo sapiens
ggcacattggcggccagggccagtccgcccggcggctcgcgcacggctccgcggtcccttttgcctgtccagccggccgcctgtccctgctccctccctccgtgaggtgtccgggttcccttcgcccagctctcccacccctacccgaccccggcgcccgggctcccagagggaactgcacttcggcagagttgaatgaatgaagagagacgcggagaactcctaaggaggagattggacaggctggactccccattgcttttctaaaaatcttggaaactttgtccttcattgaattacgacactgtccacctttaatttcctcgaaaacgcctgtaactcggctgaagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataaggtataatgggaaatgtttggccatgcttggttgttgcagttcagacaaatgtaacacacacacacatacacacacacacacacacagagacacatcttaaggggacccacaagtattgccctttaacaagacttcaaagttttctgctgtaaagaaagctgtaatatatagtaaaactaaatgttgcgtgggtggcatgagttgaagaaggcaaaggcttgtaaatttacccaatgcagtttggctttttaaattattttgtgcctatttatgaataaatattacaaattctaaaaaaaaaaaaaaaaaa

SEQ ID NO: 6 - DNA, 840 bp, Homo sapiens
ctctgctgtgctgcctcaaacgcggagggctgcgtgcagtgggagcgggctccaggagcccgagcctccagccgtcctcagagcaaggcagcaccgaggcctggccacagcaatatccatctggaagctcttcccttcactcccaactctgaggttgcctaactctttattaaaaattcagaagggggaatgccagcccctagcatggactgtgatgtttccactctggttgcctgtgtggtggatgtcgaggtctttaccaatcaggaggttaaggaaaaatttgggggactgtttcggacttatgatgactgtgtgacgttccagctatttaagagtttcagacgtgtccgtataaacttcagcaatcctaaatctgcagcccgagctaggatagagcttcatgaaacccaattcagagggaaaaaattaaagctctactttgcacaggttcagactccagagacagatggagacaaactgcacttggctccaccccagcctgccaaacagtttctcatctcgcccccttcctccccacctgttagctggcagcccatcaacgatgccacgccagtcctcaactatgacctcctctatgctgtggccaaactaggaccaggagagaagtatgagctccatgcagggactgagtccaccccaagtgtcgtcgtgcacgtgtgcgacagtgacatagaggaagaagaggacccaaagacttccccaaagccaaaaatcatccaaactcggcgtcctggcctgccaccctccgtgtccaactgagctgcctgctccttctcgataatagccgtctcctctttatcatgctttttccccctg

SEQ ID NO: 7 - DNA, 1413 bp, Homo sapiens
ccggcctcttggtactgctgaccccagccaggctacagggatcgattggagctgtccttggggctgtaattggccccagctgagcagggcaaacactgaggtcaactacaagccacaggccccttccccagcctcagttcacagctgccctgttgcagggaggcggtggcccttctgttgctagaccgagcctgtgggatataccaaggcagaggagcccatagccatgaggagcctcggggccctgctcttgctgctgagcgcctgcctggcggtgagcgctggccctgtgccaacgccgcccgacaacatccaagtgcaggaaaacttcaatatctctcggatctatgggaagtggtacaacctggccatcggttccacctgcccctggctgaagaagatcatggacaggatgacagtgagcacgctggtgctgggagagggcgctacagaggcggagatcagcatgaccagcactcgttggcggaaaggtgtctgtgaggagacgtctggagcttatgagaaaacagatactgatgggaagtttctctatcacaaatccaaatggaacataaccatggagtcctatgtggtccacaccaactatgatgagtatgccattttcctgaccaagaaattcagccgccatcatggacccaccattactgccaagctctacgggcgggcgccgcagctgagggaaactctcctgcaggacttcagagtggttgcccagggtgtgggcatccctgaggactccatcttcaccatggctgaccgaggtgaatgtgtccctggggagcaggaaccagagcccatcttaatcccgagagtccggagggctgtgctaccccaagaagaggaaggatcagggggtgggcaactggtaactgaagtcaccaagaaagaagattcctgccagctgggctactcggccggtccctgcatgggaatgaccagcaggtatttctataatggtacatccatggcctgtgagactttccagtacggcggctgcatgggcaacggtaacaacttcgtcacagaaaaggagtgtctgcagacctgccgaactgtggcggcctgcaatctccccatagtccggggcccctgccgagccttcatccagctctgggcatttgatgctgtcaaggggaagtgcgtcctcttcccctacgggggctgccagggcaacgggaacaagttctactcagagaaggagtgcagagagtactgcggtgtccctggtgatggtgatgaggagctgctgcgcttctccaactgacaactggccggtctgcaagtcagaggatggccagtgtctgtcccggggtcctgtggcaggcagcgccaagcaacctgggtccaaataaaaactaaattgtaaactcctgaaaaaaaaaaaaaaaaaaa

SEQ ID NO: 8 - DNA, 2038 bp, Homo sapiens
gcaggcccgttggaagtggttgtgacaaccccagcaatgtggagaagcctggggcttgccctggctctctgtctcctcccatcgggaggaacagagagccaggaccaaagctccttatgtaagcaacccccagcctggagcataagagatcaagatccaatgctaaactccaatggttcagtgactgtggttgctcttcttcaagccagctgatacctgtgcatcatcgaggcatctaaattagaagacctgcgagtaaaactgaagaaagaaggatattctaatatttcttatattgttgttaatcatcaaggaatctcttctcgattaaaatacacacatcttaagaataaggtttcagagcatattcctgtttatcaacaagaagaaaaccaaacagatgtctggactcttttaaatggaagcaaagatgacttcctcatatatgatagatgtggccgtcttgtatatcatcttggtttgcctttttccttcctaactttcccatatgtagaagaagccattaagattgcttactgtgaaaagaaatgtggaaactgctctctcacgactctcaaagatgaagacttttgtaaacgtgtatctttggctactgtggataaaacagttgaaactccatcgcctcattaccatcatgagcatcatcacaatcatggacatcagcaccttggcagcagtgagctttcagagaatcagcaaccaggagcaccaaatgctcctactcatcctgctcctccaggccttcatcaccaccataagcacaagggtcagcataggcagggtcacccagagaaccgagatatgccagcaagtgaagatttacaagatttacaaaagaagctctgtcgaaagagatgtataaatcaattactctgtaaattgcccacagattcagagttggctcctaggagctgatgctgccattgtcgacatctgatatttgaaaaaacagggtctgcaatcacctgacagtgtaaagaaaacctcccatctttatgtagctgacagggacttcgggcagaggagaacataactgaatcttgtcagtgacgtttgcctccagctgcctgacaaataagtcagcagcttatacccacagaagccagtgccagttgacgctgaaagaatcaggcaaaaaagtgagaatgaccttcaaactaaatatttaaaataggacatactccccaatttagtctagacacaatttcatttccagcatttttataaactaccaaattagtgaaccaaaaatagaaattagatttgtgcaaacatggagaaatctactgaattggcttccagattttaaattttatgtcatagaaatattgactcaaaccatattttttatgatggagcaactgaaaggtgattgcagcttttggttaatatgtctttttttttctttttccagtgttctatttgctttaatgagaatagaaacgtaaactatgacctaggggttttctgttggataattagcagtttagaatggaggaagaacaacaaagacatgctttccattttttcctttacttatctctcaaaacaatattactttgtcttttcaatcttctacttttaactaataaaataagtggattttgtattttaagatccagaaatacttaacacgtgaatattttgctaaaaaagcatatataactattttaaatatccatttatcttttgtatatctaagactcatcctgatttttactatcacacatgaataaaggcctttgtatctttctttctctaatgttgtatcatactcttctaaaacttgagtggctgtcttaaaagatataaggggaaagataatattgtctgtctctatattgcttagtaagtatttccatagtcaatgatggtttaataggtaaaccaaaccctataaacctgacctcctttatggttaatactattaagcaagaatgcagtacagaattggatacagtacggatttgtccaaataaattcaataaaaaccttaaaaaaaaaaaaaaaaaaaaaa

SEQ ID NO: 9 - DNA, 1050 bp, Homo sapiens
gtttgtttactcactacataaatgaccagtggcccattcagaagtctaaggacaaaatatgttgtaggactgtcctgaccactgagggacagctcacaccctgacccaacagtacactaatgcctgcagtacccaccgttcccggagtaaaccaaaagacatcctgcgactcacaaaatccattatggagttgttttaaaccttatgagaaccactgacctaggccataaaaaacaaatgaaaaaacaacaaaaactaggaatggcagtagttctgttgagttaaagaggagaaagaggagaaaccaggactggaagagatgaaattgtgagtctggaaggaaactattgcaaaggcctttattacctttaagtaaatgtctcctaactgaactgaaagccttcattctaaccattagtttggtcaggaacatttcagggacgccagggtggctgttttattttgcacttcctttactgccctcccagcagcctacctagcagtaagttccccagcctgcagtgtccccagagggcaccttccctcggctagtaacttctcagacactctggccttggggatgtcttggatcttctaggtacacagtggctgcacacagttttgccaggcctttctcaagaggtaaaagttcctagctgtttgcatttcccagaaataatgttttccatgtgtagggattttacagatttcaaagtgctttcatgtcaattactttctttaatttaaaagaagttcagataccaggtcaagctaggaatgatccggtctcagagggaaggagcgctctaggaaaggaggatcctttaatagagggccgtcctggggccgcgtgcccatggaaggcgagagtggaggagtgtcctctttctcccccaccctcaggcggcggcccggccaaagccagagggggctgtctcctcctcttccccagcagctgctgctcgctcagctcacaagccaaggccaggggacagggcggcagcgactcctctggctcccgagaagtggatccggtcgcggccactacga

SEQ ID NO: 10 - DNA, 1122 bp, Homo sapiens
caaagttttcactggagacaagtttccaggtacctacaaaagcatcacacattttgtgagttttctaggattattcttcaaggtgagaaggaagctgcagttttcaggagggtgctgtgaccctcttgagtccagataacttccttttaaagatgctcagaacatgtgatcttaggaatgaccaattgggaatgagatccaatcatttccttcattaccggcctctgcatttttttctctgagtcatgtttttaatagaaatttctcaactttggttctccctggcaaatagtcactggacttagagtacaaattatttttaaaccactaacaggatattttaaacattcctgttttgacagcttaatgctcagtgcactgaatttccccctattattcctatacatatttatcccagtgtagagaggggaaattattttgagataaacttcgaccttattttgtgtgtgtgtgtggggtgttatttaaattgcagttatgtggttcttttgaattgaggaacataagaactgcctcggagattcttacataattgcaaaggctcttcgggacactcctgtctcaggaattacctggcgattaaaacaaaataaataatgggatttaaaagtactccagatttctgatttcaaatgttttatatgaataaaaaagtacattcaattcatccaccagtaatctggactgtagatttaatcgaaatgataatttaacctcactctttccatctcctaaatgagagcatagcattgatttccacagatcctttgaaaaataggccttattctaaaatactttcctcatattgagaaactgcattcaatttctgaactgtattataagttctgaatttattcagatcagaaatggtgtgtggtcacaagctaccaaaaccaatatccttcttttatacagtgaccaatctgtgaaactagttttctaaagtaatcgtggttttcaattccctttgcctcatgcaagaatgacagaagttgttcacatgtgctgcaaattcccacgaacatacccagatttaggggaaaaaaagaaactaaaatatttcagagtgatgcatcgctacactgtgtatcccactgaaca

SEQ ID NO: 11 - DNA, 1260 bp, Homo sapiens
cggatggactccattctgccccgtatattggagtagtggagcttgggcttctctgccatggtagcagtctcctggaggtttctctaagcctgagtgaatgaatgaatgaatgaatgaataattgaaacgatagaatcaaaaatgtactttaggatgtatggttgaaaaccacaaacaatgctgaagaagaacctgccttcttcatgacggtgttggaggagttcccggaatgttttcttggctcaaattattacccagcagtggccaccctcagattccagcaaaccagtctcaagttttcactgtttaactctgaattttcttggcagcctaagaggtgagagtatgtggtaataatacatgtataggagttaatgggaagaggaagaattcaagaactaatatttactgaaaacttcctagtgatccttcctcaatgctagtccctttcaatattttatatctttaaccctccttatagtcccatgaaatgattatctccattttttcgttaatgaaatggaacatcagagaaatacaatgttcacagtcacactccggttggtgatggacatgaatatctacaccaaggactaaaatgaaatcatgcctggaagccagctgggtgaaggccctgggaacccatgaactggccatgaaaccagaggatgtcactgacagggaggaccggctgggagctaaatcactcttcagctctttggctgtgagactgcatttgatcaaaaccagaaattaggcctcagacttgtttaactgtagctagaagatccaaattctttcaagagacagagattgtttatcccttgcttcttttggaattctgtattctaactctatggggtgcattttgttttataagctggaagaagagatgttgctgcattaattttgcaatatggaaggagctagcatttgttcaacatcagtcacacactgatcattcttctaaacttctctttgttttatcctacaaaaattacctaaggttaatgtggttgttattctcattttacatttgaggatactgaggtttttaaagtaacttgctcagagtaaatgatagatctgggatccagggtcactgctattaaaaaccaaatactgagtcttattttctatgttagtgtttctcaaatatgcatggtaatctctcagcagattgtgaaatcccattactttaaaaaataaatagaaaagaaaagaatagaaaatatcagtgagcatgacatgttgaaaggt

SEQ ID NO: 12 - DNA, 1320 bp, Homo sapiens
catatttaagtcaagtgaaagagaacaaatgaatgactggcatccattaagtgagaaggaggaactaataaatgccttgtttgtaatgaaagtcattttttgcaactagtctcaaagatcctcacatacgacattgctcaatccgaaacttgaacacagccttcctgggcctacataaacgtagtctctctggatctcagaaaaataatgacccagaccacacctttcatctcttctaagttttccatgtaaagcattctccttagagccctggaaaggttactaaaattttgacccacttaatcctacaccttcagggcaacatggcattcacctgtggtttccttaatgcccaaaccagccagccaacctgttttctgccgtgttaacaaatggacaggacacaggcaccagaggttaaactttcttcctcttcccccatgtccccaggtggaccattattaccatccctattcccaagcaaccaggtagccccttcctctgtgccaagggaagaaaacaccatgcttccctctcctgagagaccttcaagagtttagagacgcctaagtccagctgtgctaaaagataaaggacaataatgcaagtctgctctgagctgccaccatagccagacatagggtagccctgaaagactagaaccaaggacagagccaaaggtgaaagaaaatatgaaaaagtgaaaacacagtatttaaacagaattttccaacagcctgcatatagtgaggacttctcaagcttctgaatccttttccattgaattgtgcaatggcacatgtatgagcaaagtcaagcctcctggctcccaggtaggcacagcccagttcttagctcctaggaagcttcagggcttaaagctccactctacttggactgtactatcaggcccccaaaatggggggagccgacagggaaggactgatttccatttcaaactgcattctggtactttgtactccagcaccattggccgatcaatatttaatgcttggagattctgactctgcgggagtcatgtcaggggaccttgggagccaatctgcttgagcttctgagtgataattattcatgggctcctgcctcttgctctttctctagcacggtcccactctgcagactcagtgccttattcagtcttctctctcgctctctccgctgctgtagccggaccctttgccttcgccactgctcagcgtctgcacatccctacaatggctaaaacagcaatgggtaaggcactgcgcctcgttctccgtcggctctacctggagcccacctctcacctcctctcttgag

SEQ ID NO: 13 - DNA, 1140 bp, Homo sapiens
gctggctccttgaggcgaggacccatactgcgcctgaacacaaggcatggctggaaatgaaacagggtgataacacactcagcctggtcaactgaacactttctcccgggctcgggtacacctggagctacaccggcagcccgcggcagtcagagagcatgtaggggcgcgagaggaaaggcaggagagagagaatgctgcagaacaacagtttagggcgtggaaagactaaccaaataaagacccagagctaaaaagctactgagggtctacactcctgggtatttccaaacatctccctcatacccccccactcatcccactcagatgagcctcttctctgaagctcagattcagcagtctctttctagaaatgaccatctagaaatgaagttgaatttcacaatgagaaagttgttcccgaaggcgatgggtcgggcaaaccttaagttacagggtttgccttgtcctgtttcttgatgtttggggagctctggagagtaaaggaaagaaatgaagttgcactaaccttcagccgagttacaggcgttttcgaggaaattaaaggtggacagtgtcgtaattcaatgaaggacaaagtttccaagatttttagaaaagcaatggggagtccagcctgtccaatctcctccctgaaatacagacacaggaagcttcagggtttcttcccgacagagattcagctgggcatatgtagactcaccaggcaggcccttccatgccttccctgtttgtctcattggaaatgagtgggaagcctttaccaaacatagcacttaaaatgaatgtataaaagaaatgacttgaaacaacagaaaataccacccattgcactgtgtaaatccttgcagagagaagatcctgcaagaggaaagctagtccatgaactcatttaacatttatgatgtaatgaactggcccctttgctacagtgtaggtggagaggtcatttccatcttaggttaattcaggacctgagtatgaagactagagtatgaaaatacagcattggaaccttctcttgcatttttttttcttgtggtgaaattgccttttcttttggtagaggggaagaaagtagaagtcaagaatataaaatgagaaaacaatcttccccct

SEQ ID NO: 14 - DNA, 1140 bp, Homo sapiens
aaccagagtggaaacatcacagtccatgctaggggctggcattcccccttctgaatttttaataaagagttaggcaacctcagagttgggagtgaagggaagagcttccagatggatattgctgtggccaggcctcggtgctgccttgctctggggacggctggaggctcgggctcctggagcccgctcccactgcacgcagccctccgcgtctgaggcagcacagcagagtaacgaacggcccggctcgctcatggcaatgacatcaccacaaagactgacacaagctgaagctatttttttttctccaagccttttatctctaggcagtgcagtggagcaaattgaacatgattatgtgctaaatctgaactcagactaaatcaattcaagcagcgttagctaggaactgagtcatagctgttgttgcagccgagtgcttatgtttgcaaaaagcaggagggggtgaatctgacaccagagtttcttctttgaggtgggggaggtgtaattctgcagatgagcctcctgaggttaaggtttgacaattttctgccttcgagatgagcaggaagttgaggcattttgcaaattgcttggcttctgttaattgctctgtgccactcagagcagccacacatgttctgggcatcctaatgcatcccgggcatgggctgaatagaaatccgttcttggagtgactaaagagctggtcgtctgtcatttagggagcatggtaagaggagataattagaggtttgtggaaattctatttgaaggctataagtgcagaccagtaacgctaagagcacccttccaagctcaggatttcaggctgtctccaaggtcattcaaaccaaaagcagttcatggaaaggagttacagcaaatatttcacacggggacagatggcagggtctaacgggcaaactcactgatgtgtctcagagcttgtcagatcctcggggcttgacttcacctgagactttgaacacatccttttgctttctcaggcttttttcctgattataaatccctgaggggcaaggccacagatgatttacctttatatcttcagtctttcacagattgcctggcacaagaagatgatacttgtttaataaataaatcaatcaaaca

SEQ ID NO: 15 - DNA, 1114 bp, Homo sapiens
agggccccgaggctcctcatggctatgggctcctctgccttggtatatcccacaggctcggtctagcaacagaagggccaccgcctccctgcaacagggcagctgtgaactgaggctggggaaggggcctgtggcttgtagttgacctcagtgtttgccctgctcagctggggccaattacagccccaaggacagctccaatcgatccctgtagcctggctggggtcagcagtaccaagaggccgggatggctgcttcagaagaggcattggccaagcacaatagggccctggagcaccaggattgggctccgccccccaaaagtcccccacagagggcatgcgaggatggggagcgacctggcctttctgctgagtcatgccatctggacctcacagctctgtgagccagcaggtcaagcctgattgggcccatttctcacaggaaaaaactgaggcccaaggagaggaagtgacttgccagagacctcagggaagtctatgggcagagccaagaccagaacccaggtatcctgtctccagagttccttctccagcccccaggcttgccctagcctttgcaaataatagagacattaacaatgatgactgttacgagcttccgttcactgagcacctgctatgtgctggctgtgtaccaggcactttacacgtgccacaggtgtccagtaaatccccacaacaagcttacgaagtaggtgctatttgtcccctttacaggcagaggagttgagtctccaagaagtgaagtgacttgcccagaatccctcagccgggagtggagtagctgggaaaggcgtggtagagaccatggacgtgggagccaggcagcctacagtggcactcactgctgtgtgaccttgggcaagtcactttacctttcagtgccttggtttcctcatctgtaaatggggataataatagttcctagctcctagcattgttgagtgagcacctgcaatgcgctaggacagtacccagcatagagtaagagctgagtgtttgtggtgggttgaacagtgccccctccagaatccatgtgcaccccgaaccgcagaatgaacctgatttgcagatgtgattagttagatgag

SEQ ID NO: 16 - DNA, 1080 bp, Homo sapiens
acattgctggggttgtcctgtaaaagagaaaacctgtcactcttcttcatagttaacttcacagtcctaactatgaatttctataaatcctgcagacataattccacttcctaaaaatacctagcccatgaattctgtctccagaaagttatgttcagactgtggcttataaaaataagtctgtgacttatgtaacattctgcaaacacaacctaacttttgtttgagacacaccaagttctgactgttctttctatcctccaagaagaaagggataggccgggtgtggtggctcacgcctgtaatcccagcactttgggaggccgaggctggcagatcacgatgtcaggagatcgagaccatcctggctaacacggtgaaaccccttctctactaaaattatacaaaaaaaaaaaaaaaattagccaggcttggtggcagacacctgtagtcccagctactcaggaggctgaggcaggagaatggtgtgaacccgggcggtggagcttgcagtgagctgagatcgcgcccctgcactccagcctgggcgacagaacgagactctgtctcaaaaaaaaaaaaacaaaagaagaagaagaaagggataaatagagcattctgcacagaaatgaaaagagccagcaaaaaaagagaaccaaggaaaaaagatgatggcagaaaagacagtataccaacatgaatgtggctcatgtcgggcccttagctcttcaagttcaaacattctatatttatctcttgggtatggctcccttttgtttctcttattccttgaagtctggctgtatgtatctaaccaagtagaaatatcacacatctgccccctctaccatttaagactctttgaaatccacactcaattagcctatttattggaaagttcctatgactagaaaattcctatgactagacagcactttctttggtaaaaagatgcttcctctgagcaacgaaattaaaatgcttttaaatacctgactagaaatatcctcaaaactagggctagctctctaaacagaaatgttcaaaaacaggtgaactgtagacaggtgataaagaaaa

SEQ ID NO: 17 - DNA, 21 bp, Artificial Sequence
  Synthetic Construct
ccagtggcccattcagaagtc

SEQ ID NO: 18 - DNA, 20 bp, Artificial Sequence
  Synthetic Construct
ccacttctcgggagccagag

SEQ ID NO: 19 - DNA, 22 bp, Artificial Sequence
  Synthetic Construct
ggatacacagtgtagcgatgca

SEQ ID NO: 20 - DNA, 21 bp, Artificial Sequence
  Synthetic Construct
gctgcagttttcaggagggtg

SEQ ID NO: 21 - DNA, 20 bp, Artificial Sequence
  Synthetic Construct
gcagtgaccctggatcccag

SEQ ID NO: 22 - DNA, 21 bp, Artificial Sequence
  Synthetic Construct
ggtagcagtctcctggaggtt

SEQ ID NO: 23 - DNA, 21 bp, Artificial Sequence
  Synthetic Construct
ccttcctctgtgccaagggaa

SEQ ID NO: 24 - DNA, 20 bp, Artificial Sequence
  Synthetic Construct
ggatgtgcagacgctgagca

SEQ ID NO: 25 - DNA, 22 bp, Artificial Sequence
  Synthetic Construct
cctaagatggaaatgacctctc

SEQ ID NO: 26 - DNA, 21 bp, Artificial Sequence
  Synthetic Construct
gggtgataacacactcagcct

SEQ ID NO: 27 - DNA, 21 bp, Artificial Sequence
  Synthetic Construct
gctcttagcgttactggtctg

SEQ ID NO: 28 - DNA, 21 bp, Artificial Sequence
  Synthetic Construct
ggcaacctcagagttgggagt

SEQ ID NO: 29 - DNA, 20 bp, Artificial Sequence
  Synthetic Construct
cctagcgcattgcaggtgct

SEQ ID NO: 30 - DNA, 21 bp, Artificial Sequence
  Synthetic Construct
cctctgccttggtatatccca

SEQ ID NO: 31 - DNA, 21 bp, Artificial Sequence
  Synthetic Construct
cgttgctcagaggaagcatct

SEQ ID NO: 32 - DNA, 22 bp, Artificial Sequence
  Synthetic Construct
cctagcccatgaattctgtctc

(19) United States
(12) Patent Application Publication
(10) Pub. No.: US 2006/0068402 A1
(43) Pub. Date: Mar. 30, 2006

(54) Methylated promoters of colon cancer-specific expression-decreased genes and use thereof

(75) Inventors: Sungwhan An, Daejeon (KR); ChiWang Yoon, Daejeon (KR); TaeJeong Oh, Daejeon (KR); DaeKyoung Yoon, Daejeon (KR); SunWoo Lee, Daejeon (KR); MyungSoon Kim, Daejeon (KR); SukKyung Woo, Daejeon (KR)

Correspondence Address: INTELLECTUAL PROPERTY / TECHNOLOGY LAW, PO BOX 14329, RESEARCH TRIANGLE PARK, NC, 27709, US

(73) Assignee: GENOMICTREE, INC., DAEJEON (KR)

(21) Appl. No.: 10/984,481

(22) Filed: Nov. 9, 2004

(30) Foreign Application Priority Data
Sep. 24, 2004 (KR) 10-2004-0076765

(51) Int. Cl. C12Q 1/68; C07H 21/04

(52) U.S. Cl. 435/6; 536/23.2

(57) ABSTRACT

Methylated promoters of colon cancer-specific genes and use thereof. Various disclosed aspects of the invention include methylated promoters of the colon cancer specific expression-decreased genes, microarrays for cancer diagnosis on which the methylated promoters are immobilized, and cancer diagnosis kits containing the methylated promoters. The methylated promoters of colon cancer-specific expression-decreased genes have utility for early detection of cancer and as targets for screening new drugs useful in the early treatment of cancer.

### BACKGROUND OF THE INVENTION

1. Field of the Invention

The present invention generally relates to the methylated promoters of colon cancer-specific genes and the use thereof. More specifically, the invention relates in various aspects to methylated promoters of colon cancer-specific expression-decreased genes, to a microarray for cancer diagnosis on which the methylated promoter is immobilized, and to a cancer diagnostic kit containing the methylated promoter.

2. Background of the Related Art

Despite the current developed state of medical science, five-year survival rates of human cancers, particularly solid cancers (cancers other than blood cancer) that account for a large majority of human cancers, are less than 50%. About two-thirds of all cancer patients are detected at a progressed stage, and most of them die within two years after the diagnosis of cancer. Such poor results in cancer diagnosis and therapy are due not only to the problem of therapeutic methods, but also to the fact that it is not easy to diagnose cancer at an early stage or to accurately diagnose progressed cancer or observe it following therapeutic intervention.

In current clinical practice, the diagnosis of cancer typically is confirmed by performing tissue biopsy after history taking, physical examination and clinical assessment, followed by radiographic testing and endoscopy if cancer is suspected. However, the diagnosis of cancer by existing clinical practices is possible only when the number of cancer cells is more than a billion, and the diameter of cancer is more than 1 cm. In this case, the cancer cells already have metastatic ability, and at least half thereof have already metastasized. Meanwhile, tumor markers for monitoring substances that are directly or indirectly produced from cancers, are used in cancer screening, but they cause confusion due to limitations in accuracy, since up to about half thereof appear normal even in the presence of cancer, and they often appear positive even in the absence of cancer. Furthermore, the anticancer agents that are mainly used in cancer therapy have the problem that they show an effect only when the volume of cancer is small.

The reason why the diagnosis and treatment of cancer are difficult is that cancer cells have many differences from normal cells and are highly complex and variable. Cancer cells grow excessively and continuuously in their own way, continually survive without death, invade surrounding tissues and are diffused (metastasized) to distal organs, thereby causing human beings to die. Despite the attack of an immune mechanism or anticancer therapy, cancer cells survive and continually develop, and cell groups that are most suitable for survival selectively propagate. Cancer cells are living bodies with a high degree of viability, which occur by the mutation of a large number of genes. In order that one cell is converted to a cancer cell and developed to a malignant cancer lump that is detectable in clinics, the mutation of a large number of genes must occur. Thus, in order to diagnose and treat cancer at the root, approaches at a gene level are necessary.

Recently, genetic analysis is actively being attempted to diagnose cancer. The simplest typical method is to detect the presence of ABL: BCR fusion genes (the genetic characteristic of leukemia) in blood by PCR. This method has an accuracy of more than 95%, and after the diagnosis and therapy of chronic myelocytic leukemia using this simple and easy genetic analysis, this method is being used for the assessment of the result and follow-up study, etc. However, this method has the deficiency that it can be applied only to some blood cancers.

Another method is being attempted, in which the presence of genes expressed by cancer cells is detected by RT-PCR and blotting, thereby diagnosing cancer cells present in blood cells. However, this method has the deficiency that it can be applied only to some cancers, including prostate cancer and melanoma, and has a high false positive rate. Additionally, it is difficult to standardize detection and reading in this method, and its utility is also limited (Kopreski, M. S. et al., Clin. Cancer Res., 5:1961, 1999; Miyashiro, I. et al., Clin. Chem., 47:505, 2001).

Recently, genetic testing using a DNA in serum or plasma is actively being attempted. This is a method of detecting a cancer-related gene that is isolated from cancer cells and released into blood and present in the form of a free DNA in serum. It is found that the concentration of DNA in serum is increased by a factor of 5-10 times in actual cancer patients as compared to that of normal persons, and such increased DNA is released mostly from cancer cells. The analysis of cancer-specific gene abnormalities, such as the mutation, deletion and functional loss of oncogenes and tumor-suppressor genes, using such DNAs isolated from cancer cells, allows the diagnosis of cancer. In this effort, there has been an active attempt to diagnose lung cancer, head and neck cancer, breast cancer, colon cancer, and liver cancer, etc., by examining the promoter methylation of mutated K-Ras oncogenes, p53 tumor-suppressor genes and p16 genes in serum, and the labeling and instability of microsatellite (Chen, X. Q. et al., Clin. Cancer Res., 5:2297, 1999; Esteller, M. et al., Cancer Res., 59:67, 1999; Sanchez-Cespedes, M. et al., Cancer Res., 60:892, 2000; Sozzi, G. et al., Clin. Cancer Res., 5:2689, 1999).

Meanwhile, in samples other than blood, the DNA of cancer cells can also be detected. A method is being attempted in which the presence of cancer cells or oncogenes in sputum or bronchoalveolar lavage of lung cancer patients is detected by a gene or antibody test (Palmisano, W. A. et al., Cancer Res., 60:5954, 2000; Sueoka, E. et al., Cancer Res., 59:1404, 1999). Additionally, other methods of detecting the presence of oncogenes in feces of colon and rectal cancer patients (Ahlquist, D. A. et al., Gastroenterol., 119:1219, 2000) and detecting promoter methylation abnormalities in urine and prostate fluid (Goessl, C. et al., Cancer Res., 60:5941, 2000) are being attempted. However, in order to accurately diagnose cancers that cause a large number of gene abnormalities and show various mutations characteristic of each cancer, a method, by which a large number of genes are simultaneously analyzed in an accurate and automatic manner, is required. However, such a method is not yet established.

Accordingly, methods of diagnosing cancer by the measurement of DNA methylation are being proposed. When the promoter CpG island of a certain gene is over-methylated, the expression of such a gene is silenced. This is interpreted to be a main mechanism by which the function of this gene is lost even when there is no mutation in the protein-coding sequence of the gene in a living body. Also, this is analyzed as a factor by which the function of a number of tumor-suppressor genes in human cancer is lost. Thus, detecting the methylation of the promoter CpG island of tumor-suppressor genes is greatly needed for the study of cancer. Recently, an attempt has actively been conducted to determine promoter methylation, by methods such as methylation-specific PCR (hereinafter, referred to as MSP) and automatic DNA sequencing, for diagnosis and screening of cancer.

A significant number of diseases are caused by genetic abnormalities, and the most frequent forms of genetic abnormalities are changes in gene-coding sequences. Such genetic changes are called mutations. When there are mutations in any gene, the structure and function of a protein coded by such a gene are changed, and hindrance and deletion are caused, and such a mutated protein causes a disease. However, even if there are no mutations in a certain gene, an abnormality in the expression of this gene can cause disease. A typical example is methylation where methyl groups are attached to gene transcriptional regulatory sites, e.g., the cytosine base sites of CpG islands, in which case the expression of this gene is blocked. This is called an epigenetic change, which is transferred to offspring cells in a similar manner to mutations, and causes the same effect, i.e., the loss of expression of the corresponding protein. The most typical change is that the expression of tumor-suppressor genes is blocked by the methylation of promoter CpG islands in cancer cells, and this blocked expression is an important mechanism of causing cancer (Robertson, K. D. & Jones, P. A., Carcinogensis, 21:461, 2000).

For the accurate diagnosis of cancer, it is important to detect not only a mutated gene but also to determine a mechanism, where the mutation of this gene appears. While previous studies have been conducted by focusing on the mutations of a coding sequence, i.e., micro-changes, such as point mutations, deletions and insertions, or macroscopic chromosomal abnormalities, recently, epigenetic changes are reported to be as important as these mutations, and a typical example of such epigenetic changes is the methylation of promoter CpG islands.

In the genomic DNA of mammal cells, there is a fifth base in addition to A, C, G and T, namely, 5-methylcytosine, in which a methyl group is attached to the fifth carbon of the cytosine ring (5-mC). 5-mC is always attached only to the C of a CG dinucleotide (5′-mCG-3′), which is frequently marked CpG. The C of CpG is mostly methylated by attachment with a methyl group. The methylation of this CpG inhibits a repetitive sequence in genomes, such as alu or transposon, from being expressed. Also, this CpG is a site where an epigenetic change in mammalian cells appears most often. The 5-mC of this CpG is naturally deaminated to T, and thus, the CpG in mammal genomes shows only 1% of frequency, which is much lower than a normal frequency (¼× 1/4=6.25%).

Regions in which CpG is exceptionally integrated are known as CpG islands. The CpG islands refer to sites which are 0.2-3 kb in length, and have a C+G content of more than 50% and a CpG ratio of more than 3.75%. There are about 45,000 CpG islands in the human genome, and they are mostly found in promoter regions regulating the expression of genes. Actually, the CpG islands occur in the promoters of housekeeping genes accounting for about 50% of human genes (Cross, S. H. & Bird, A. P., Curr. Opin. Gene Develop., 5:309, 1995).

In the somatic cells of normal persons, the CpG islands of such housekeeping gene promoter sites are un-methylated, but imprinted genes and the genes on inactivated X chromosomes are methylated such that they are not expressed during development.

During a cancer-causing process, methylation is found in promoter CpG islands, and the restriction on the corresponding gene expression occurs. Particularly, if methylation occurs in the promoter CpG islands of tumor-suppressor genes that regulate cell cycle or apoptosis, restore DNA, are involved in the adhesion of cells and the interaction between cells, and/or suppress cell invasion and metastasis, such methylation blocks the expression and function of such genes in the same manner as the mutations of a coding sequence, thereby promoting the development and progression of cancer. In addition, partial methylation also occurs in the CpG islands according to aging.

An interesting fact is that, in the case of genes whose mutations are attributed to the development of cancer in congenital cancer but do not occur in acquired cancer, the methylation of promoter CpG islands occurs instead of mutation. Typical examples include the promoter methylation of genes, such as acquired renal cancer VHL (von Hippel Lindau), breast cancer BRCA1, colon cancer MLH1, and stomach cancer E-CAD. In addition, in about half of all cancers, the promoter methylation of p16 or the mutation of Rb occurs, and the remaining cancers show the mutation of p53 or the promoter methylation of p73, p14 and the like.

An important fact is that an epigenetic change caused by promoter methylation causes a genetic change (i.e., the mutation of a coding sequence), and the development of cancer is progressed by the combination of such genetic and epigenetic changes. In a MLH1 gene as an example, there is the circumstance in which the function of one allele of the MLH1 gene in colon cancer cells is lost due to its mutation or deletion, and the remaining one allele does not function due to promoter methylation. In addition, if the function of MLH1, which is a DNA restoring gene, is lost due to promoter methylation, the occurrence of mutation in other important genes is facilitated to promote the development of cancer.

Most cancers show three common characteristics with respect to CpG, namely, hypermethylation of promoter CpG islands of tumor-suppressor genes, hypomethylation of the remaining CpG base sites, and an increase in the activity of methylation enzyme, namely, DNA cytosine methyltransferase (DNMT) (Singal, R. & Ginder, G. D., Blood, 93:4059, 1999; Robertson, K. & Jones, P. A., Carcinogensis, 21:461, 2000; Malik, K. & Brown, K. W., Brit. J. Cancer, 83:1583, 2000).

When promoter CpG islands are methylated, the reason why the expression of the corresponding genes is blocked is not clearly established, but is presumed to be because a methyl CpG-binding protein (MECP) or a methyl CpG-binding domain protein (MBD), and histone deacetylase, bind to methylated cytosine thereby causing a change in the chromatin structure of chromosomes and a change in histone protein.

There is dispute about whether the methylation of promoter CpG islands directly causes the development of cancer or is a secondary change after the development of cancer. However, it is clear that the promoter methylation of tumor-related genes is an important index to cancer, and thus, can be used in many applications, including the diagnosis and early detection of cancer, the prediction of the risk of the development of cancer, the prognosis of cancer, follow-up examination after treatment, and the prediction of a response to anticancer therapy. Recently, an attempt to examine the promoter methylation of tumor-related genes in blood, sputum, saliva, feces or urine and to use the examined results for the diagnosis and treatment of various cancers, has been actively conducted (Esteller, M. et al., Cancer Res., 59:67, 1999; Sanchez-Cespedez, M. et al., Cancer Res., 60:892, 2000; Ahlquist, D. A. et al., Gastroenterol., 119:1219, 2000).

In order to maximize the accuracy of cancer diagnosis using promoter methylation, analyze the development of cancer according to each stage and discriminate a change according to cancer and aging, an examination that can accurately analyze the methylation of all the cytosine bases of promoter CpG islands is required. Currently, a standard method for this examination is a bisulfite genome-sequencing method, in which a sample DNA is treated with sodium bisulfite, and all regions of the CpG islands of a target gene to be examined are amplified by PCR, and then, the base sequence of the amplified regions is analyzed. However, this examination has the problem that there are limitations on the number of genes or samples that can be examined at a given time. Other problems are that automation is difficult, and much time and expense are required.

In Johnson Hopkins University, MD Anderson Cancer Center and Medical University of Berlin, etc., studies on the promoter methylation of cancer-related genes are being actively conducted. The fundamental data thus obtained are disseminated through the DNA Methylation Society (DMS) and stored in MethDB (http://www.methdb.de). Meanwhile, EpiGenX Pharmaceuticals, Inc. is now developing therapeutic agents associated with the methylation of CpG islands, and Epigenomics, Inc. is now conducting studies to apply promoter methylation to cancer diagnosis by examining promoter methylation using various techniques, such as DNA chips and MALDI-TOF.

The methylation of promoter CpG islands has a deep connection with physiological phenomena, such as the development and differentiation of a human body, and also aging, the development of various cancers and positive diseases. Particularly, the methylation of the promoter CpG islands of tumor-related genes can act as an index of cancer since they play an important role in the development and progression of cancer. However, in the existing method, there are difficulties in that all the CpG islands of many genes should be amplified by MSP, and analyzed by a base sequence analysis method (bisulfite genome-sequencing method). Furthermore, there is no method that can analyze various changes of the promoter methylation of many genes at a given time in an accurate, rapid and automatic manner, and can be applied to the diagnosis, early diagnosis or assessment of each stage of various cancers in clinical practice.

In the area of screening of new cancer suppressor genes associated with methylation, many studies have been performed. Examples of the existing screening methods include: a method where the genomic DNAs of cancer tissues and normal tissues are restricted with methylation-related restriction enzymes, and many DNA fragments obtained are all cloned, and then DNA fragments having the difference between cancer tissues and normal tissues are selected, sequenced and screened; and a method using a binding column that recognizes CpG islands (Huang, T. H. et al., Hum. Mol. Genet., 8:459, 1999; Cross, S. H. et al., Nat. Genet., 6:236, 1994). However, such methods have shortcomings in that they require much time, are not efficient to screen gene candidates and also are difficult to apply in actual clinical practice.

Accordingly, the present inventors have conducted extensive studies and have screened methylated promoters of colon cancer-specific expression-decreased genes from colon cancer tissues and cell lines, and confirmed the methylated promoters as useful for cancer diagnosis, thereby perfecting the present invention.

### SUMMARY OF THE INVENTION

The present invention relates to the methylated promoters of the colon cancer-specific expression-decreased genes, as hereinafter more fully described.

The present invention also relates to a microarray useful for the early detection of cancer, on which the methylated promoters are immobilized.

The present invention also relates to a cancer diagnosis kit containing the methylated promoters.

The present invention also relates to a primer mixture useful for the amplification of a clinical sample-derived DNA for the early detection of cancer.

The present invention also relates to a method for the early detection of cancer, which is characterized by the use of the microarray and/or the primer mixture.

Other aspects, features and advantages of the invention will be more fully apparent from the ensuing disclosure and appended claims.

### BRIEF DESCRIPTION OF DRAWINGS

FIG. 1 shows the results of direct comparison test and indirect comparison test on the changes of gene expressions in 10 kinds of colon cancer clinical samples and 10 kinds of normal colon samples using 17K cDNA microarray and gene expression patterns of 8 kinds of subject genes.

FIG. 2 shows the results of cDNA microarray which expression changes of 8 kinds of genes were determined, after treating colon cancer cell line (HCT116) with DAC alone and DAC+TSA, so as to confirm that expression of 8 kinds of genes screened in the present invention is controlled by promoter methylation.

FIG. 3 shows the results of an HpaII/MspI enzyme digestion assay for 8 kinds of genes cloned in the present invention, in which the enzyme digestion assay can measure whether promoter methylation occurs.

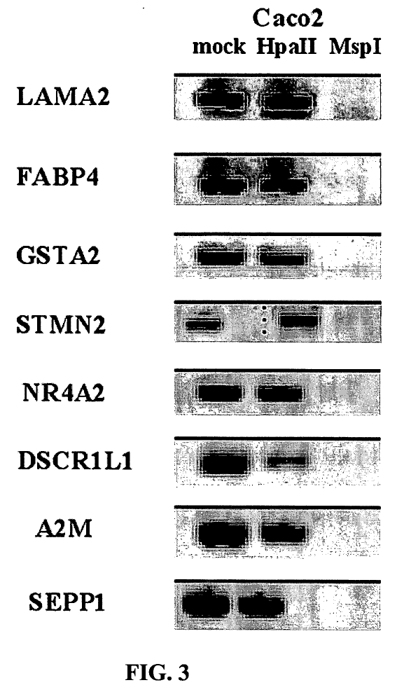

FIG. 4 shows the promoter methylation profiles on 2 colon cancer cell lines (HCT 116; Caco2), using a microarray on which the methylated promoters of 8 genes screened in the present invention are attached.

FIG. 5 shows the results of performing bisulfite sequencing on colon cancer cell line HCT116, to confirm the promoter methylation of 3 genes (NR4A2, A2M and SEPP1).

FIG. 6 shows promoter methylation profiles on the cancer tissues from 17 colon cancer patients and the lymphocyte from 3 normal persons, using a microarray on which the methylated promoters of 8 genes screened in the present invention are immobilized.

### DETAILED DESCRIPTION OF THE INVENTION, AND PREFERRED EMBODIMENT THEREOF

In the present invention, eight colon cancer-specific expression-decreased genes associated with methylation were screened, and their methylated promoters were identified, thereby confirming that they can be clinically applied.

In one aspect, the present invention provides a methylated promoter of the colon cancer-specific expression-decreased gene selected from the group consisting of LAMA2, FABP4, GSTA2, STMN2, NR4A2, DSCR1L1, A2M and SEPP1.

In the present invention, said LAMA2, FABP4, GSTA2, STMN2, NR4A2, DSCR1L1, A2M and SEPP1 preferably have DNA sequence of SEQ ID NO: 1 to SEQ ID NO: 8, respectively. The methylated promoter preferably contains at least one methylated CpG dinucleotide, and more preferably comprises any one DNA sequence selected from the group consisting of:

(a) 766-805 region of SEQ ID NO: 9;

(b) 896-935 region of SEQ ID NO: 9;

(c) 201-240 region of SEQ ID NO: 10;

(d) 206-245 region of SEQ ID NO: 11;

(e) 556-595 region of SEQ ID NO: 11;

(f) 661-700 region of SEQ ID NO: 11;

(g) 1176-1215 region of SEQ ID NO: 12;

(h) 81-120 region of SEQ ID NO: 13;

(i) 121-160 region of SEQ ID NO: 13;

(j) 226-265 region of SEQ ID NO: 14;

(k) 646-685 region of SEQ ID NO: 14;

(l) 226-265 region of SEQ ID NO: 15;

(m) 776-815 region of SEQ ID NO: 15;

(n) 266-305 region of SEQ ID NO: 16; and

(o) 476-515 region of SEQ ID NO: 16.

The methylated promoter has more preferably any one DNA sequence selected from the group consisting of SEQ ID NO: 9 to SEQ ID NO: 16.

In another aspect, the present invention provides a microarray for cancer detection, in which the methylated promoter is immobilized on a solid substrate, as well as a cancer diagnosis kit containing the methylated promoter.

In still another aspect, the present invention provides a primer mixture containing at least one primer pair selected from the group consisting of SEQ ID NOs: 17/18, SEQ ID NOs: 19/20, SEQ ID NOs: 21/22, SEQ ID NOs: 23/24, SEQ ID NOs: 25/26, SEQ ID NOs: 27/28, SEQ ID NOs: 29/30, and SEQ ID NOs: 31/32.

In yet another aspect, the present invention provides a method for detecting the promoter methylation of a clinical cancer sample-derived gene, the method including the steps of: (a) isolating sample DNAs from a clinical sample; (b) treating the isolated sample DNAs with (i) an agent that modifies nonmethylated cytosine residues, or (ii) any one or more methylation sensitive restriction enzyme selected from the group consisting of HpaII, MspI, BssHII, BstUI and NotI; (c) amplifying the treated DNAs using a primer capable of amplifying CpG island which is originated from the promoter of the gene selected from the group consisting of LAMA2, FABP4, GSTA2, STMN2, NR4A2, DSCR1L1, A2M and SEPP1; and (d) determining promoter methylation according to the presence of the amplified product in the step (c).

In still another aspect, the present invention provides a method for diagnosing cancer, the method comprises detecting methylation of CpG-containing promoter of the gene selected from the group consisting of LAMA2, FABP4, GSTA2, STMN2, NR4A2, DSCR1L1, A2M and SEPP1, in a clinical sample-derived DNA. In the method for diagnosing cancer, the detecting comprises the steps of: (a) isolating sample DNAs from a clinical sample; (b) treating the isolated sample DNAs with (i) an agent that modifies nonmethylated cytosine residues, or (ii) any one or more methylation sensitive restriction enzyme selected from the group consisting of HpaII, MspI, BssHII, BstUI and NotI; (c) amplifying the treated DNAs using a primer capable of amplifying CpG island, which is originated from the promoter of the gene selected from the group consisting of LAMA2, FABP4, GSTA2, STMN2, NR4A2, DSCR1L1, A2M and SEPP1; and (d) determining promoter methylation according to the presence or absence of the amplified product in the step (c).

In the practice of the present invention, the agent that modifies nonmethylated cytosine residues can be of any suitable type, but preferably is bisulfite. The method, wherein nonmethylated cytosine residues are modified using bisulfite to detect methylation, has been described in detail in the prior art (WO 01/26536; and U.S. 2003/0148326A1).

Typically, the method of amplifying is by PCR, as described herein and as is commonly used by those of ordinary skill in the art. The amplified products are preferably identified as methylated or non-methylated by sequencing. Sequences amplified by the method of the present invention can be further evaluated, detected, cloned, sequenced, and the like, either in solution or after being bound to a solid support, by any detection method of a specific DNA sequence such as PCR, oligomer restriction, allele-specific oligonucleotide (ASO) probe analysis (Conner, B. J. et al., PNAS, 80:278, 1983; Saiki, R. K. et al., Nature, 324:163, 1986), oligonucleotide ligation assays (OLAs) (Landegren, U. et al., Science, 241:1077, 1988; Landegren, U., Bioessays, 15:761, 1993), and the like.

Optionally, the methylation pattern of the nucleic acid can be confirmed by restriction enzyme digestion and Southern blot analysis. Examples of methylation sensitive restriction enzymes that can be used to detect 5′CpG methylation include SmaI, SacII, EagI, HpaII, MspI, BssHII, BstUI and NotI. Any restriction enzyme that includes CG as part of its recognition site inhibits the C from being methylated. Typically, the methylation sensitive restriction enzyme is HpaII, MspI, or BssHII, used alone or in combination. Other methylation sensitive restriction enzymes are well known to those of skill in the art and can be used to advantage in the broad practice of the invention.

The primer capable of amplifying CpG island is preferably a primer mixture containing at least one primer pair selected from the group consisting of SEQ ID NOs: 17/18, SEQ ID NOs: 19/20, SEQ ID NOs: 21/22, SEQ ID NOs: 23/24, SEQ ID NOs: 25/26, SEQ ID NOs: 27/28, SEQ ID NOs: 29/30, and SEQ ID NOs: 31/32. In a more preferable embodiment of the present invention, the primer mixture includes all primers of SEQ ID NOs: 17 to 32.

In one embodiment of the present invention, the step (d) can be carried out by electrophoresing the amplified product obtained in step (c), and determining presence of methylation, where the PCR product is present in the DNA treated with HpaII, and absence of methylation, where the PCR product is absent in the DNA treated with HpaII, under the state where the PCR product is present in the mock DNA. Further, step (d) may also include hybridizing the amplified product of step (c) with a microarray for cancer detection in accordance with present invention, and determining the presence of methylation, where hybridization has occurred in both the mock DNA and the DNA treated with HpaII.

In the present invention, the term ‘clinical sample’ refers to sputum, feces, urine, cell membrane, encephalon, amniotic fluid, eyeball, intestines, and blood, etc. as well as a tissue and cell derived from subjects to be diagnosed on whether they have cancer. The term ‘colon cancer specific expression-decreased genes’ refers to genes whose expression is specifically suppressed in colon cancer. The term ‘gene’ in such context refers to DNA sequences that code for or correspond to a particular sequence of ribonucleic acids or amino acids that comprise all or part of one or more proteins. The term ‘mock DNA’ refers to a DNA isolated from clinical samples with no treatment. A “promoter sequence” is a DNA regulatory region capable of binding RNA polymerase and initiating transcription of a downstream (3′ direction) coding sequence.

### EXAMPLES

The present invention will hereinafter be described in further detail by examples. It will however be obvious to a person skilled in the art that these examples can be modified into various different forms and the present invention is not limited to or by the examples. These examples are presented to further illustrate the present invention.

### Example 1

### Screening of Colon Cancer-Specific Expression-Decreased Genes

To screen the genes whose expression is specifically suppressed in colon cancer, human 17K cDNA microarray tests were performed by direct comparison method and in direct comparison method using 10 colon cancer tissue samples and 10 normal colon samples.

As a direct comparison method, after labeling 100 μg of total RNA, isolated from colon cancer tissue samples, and 100 μg of total RNA, isolated from normal colon tissue samples corresponding to cancer tissues, with fluorescence dye Cy5-dUTP and Cy3-dUTP respectively, they were hybridized in human 17K cDNA microarray (GenomicTree, Inc., Korea), thereby analyzing the changes of the expressions.

As a indirect comparison method, 100 μg of total RNA, isolated from 10 kinds of colon cancer tissue samples and 10 kinds of normal colon samples, was labeled with fluorescence dye Cy5-dUTP and 100 μg of common reference RNA (Korean Patent Application Number 10-2003-0058033), mixed with total RNA isolated from 13 kinds of cell lines, was labeled with fluorescence dye Cy3-dUTP and then subjected to hybridizing in human 17K cDNA microarray (GenomicTree, Inc., Korea), followed by analysis of the changes of the expressions.

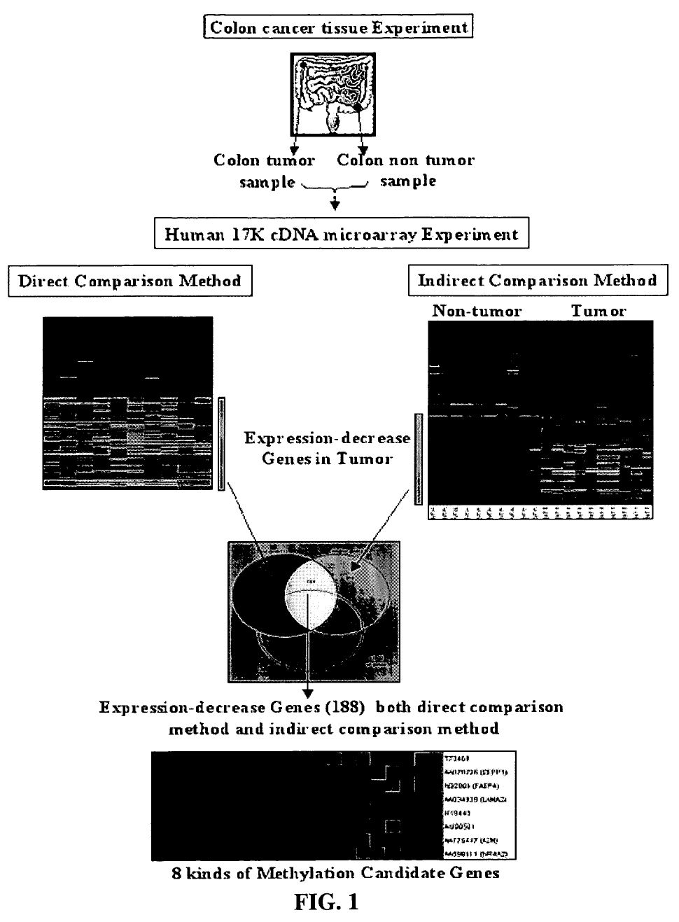

The analysis of the changes of gene expressions in colon cancer tissue samples and normal colon samples by the direct comparison method and the indirect comparison method resulted in obtaining 188 genes whose expression was decreased in both methods, and 8 kinds of genes (colon cancer-specific expression-decreased genes), whose expression was suppressed by promoter methylation, were screened from the 188 genes obtained by the above method (FIG. 1 and Table 1).

TABLE 1
8 kinds of colon cancer specific down-regulated genes

| | SEQ | |
|---|---|---|
| Name | ID NO: | Full name |
| LAMA2 | 1 | laminin alpha2(merosin, congenital) |
| FABP4 | 2 | Fatty acid binding protein 4, adipocyte |
| GSTA2 | 3 | glutathione S transferase A2 |
| STMN2 | 4 | stathmin-like 2 |
| NR4A2 | 5 | nuclear receptor subfamily 4 group A, member 2 |
| DSCR1L1 | 6 | Down syndrome candidate region 1-like |
| A2M | 7 | alpha-2-macroglobulin |
| SEPP1 | 8 | selenoprotein P, plasma, 1 |

### Example 2

### Expression Control of the 8 Genes by Promoter Methylation

Cell line was treated with DAC (5′-aza-deoxycytidine) which is a demethylating agent that suppresses methylation at the promoter and HDAC (histone deacetylase) protein specific suppressor, TSA (trichostatin A) which suppresses gene expressions in another pathway, to detect that the expression of 8 kinds of genes screened in Example 1 is controlled by methylation of their promoters.

Colon cancer cell line, HCT116 (ATCC CCL 247) was cultured, treated with DAC and/or TSA, and then total RNAs were extracted from the treated HCT116 and the control group (a group without agent treatment), followed by performing 17K cDNA microarray assay. First, cell line was suspended in a DMEM medium containing 10% FBS, 1% penicillin, 1% streptomycin and cultured under the condition of 37° C., 5% CO₂. when the cell line was cultured, it was divided into 4 groups, and then after treating by adding 1 μl of agent into each l ml of culture broth in the same condition as the one in the Table 2, cell line was collected to isolate total RNA.

TABLE 2

| | DAC | | DAC + TSA | |
|---|---|---|---|---|
| | treated | | treated | |
| | group | Control | group | Control |
| 1 day | DAC 200 nM | 50% acetic | DAC 200 nM | 50% acetic |
| | | acid | | acid |
| 2 days | DAC 200 nM | 50% acetic | DAC 200 nM | 50% acetic |
| | | acid | | acid |
| 3 days | DAC 200 nM | 50% acetic | TSA 300 nM | 100% ethanol |
| | | acid | | |

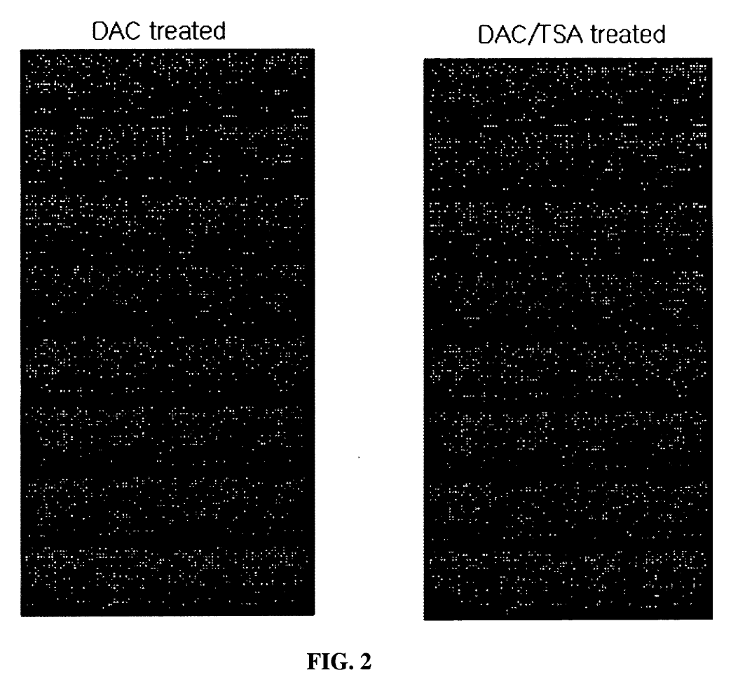

After labeling total RNAs, from a gene group treated with DAC alone and a gene group treated with DAC+TSA, with Cy5 fluorescence dye and labeling total RNA, from a control group corresponding to the above, with Cy3 flouorescence dye, a test was performed by 17K cDNA chip (GenomicTree, Inc., Korea). The total RNAs were hybridized at 65° C. for 16 hours and washed, and then scanned with Axon Scanner 4000B and GenePixPro 4.0 program (FIG. 2).

In this assay, genes whose expression is suppressed by methylation of promoters are expected to be re-expressed since the promoter methylation phenomenon disappears when the assay such as the above one is conducted. The result of the assay suggests that the expressions of 8 kinds of genes are significantly increased in cell lines treated with methylation suppressor, as compared to the control group (Table 3), thereby confirming that the expressions of 8 kinds of genes according to the present invention are controlled by promoter methylation.

TABLE 3
The expression changes of 8 kinds of genes in colon
cancer cell line by methylation suppressor treatment

| Name | Ratio of median | Cy5 signal | Cy3 signal |
|---|---|---|---|
| LAMA2 | 21.163 | 14708 | 695 |
| FABP4 | 10.105 | 1152 | 114 |
| GSTA2 | 2.854 | 839 | 294 |
| STMN2 | 2.827 | 2092 | 740 |
| NR4A2 | 2.275 | 1151 | 506 |
| DSCR1L1 | 1.642 | 1441 | 878 |
| A2M | 1.805 | 213 | 118 |
| SEPP1 | 1.175 | 2120 | 1804 |

### Example 3

### Cloning of the Promoters of 8 Genes and Digestion Assay

Genomic DNAs obtained from lymphocyte of normal person were subjected to PCR amplification [(each consisting of 1 min at 94° C.→1 min at 66° C.→1 min at 72° C.) 30 cycles] as a template using primer of below Table 4 to clone the promoters of 8 kinds of genes screened in Example 1 and proved in Example 2.

TABLE 4

Primers used in cloning
the promoters of 8 kinds of genes

| | | |
|---|---|---|
| LAMA2 | SEQ ID NO: 17 | 5′-cca gtg gcc cat tca gaa gtc-3′ |
| | SEQ ID NO: 18 | 5′-cca ctt ctc ggg agc cag ag-3′ |
| FABP4 | SEQ ID NO: 19 | 5′-gga tac aca gtg tag cga tgc a-3′ |
| | SEQ ID NO: 20 | 5′-gct gca gtt ttc agg agg gtg-3′ |
| GSTA2 | SEQ ID NO: 21 | 5′-gca gtg acc ctg gat ccc ag-3′ |
| | SEQ ID NO: 22 | 5′-ggt agc agt ctc ctg gag gtt-3′ |
| STMN2 | SEQ ID NO: 23 | 5′-cct tcc tct gtg cca agg gaa-3′ |
| | SEQ ID NO: 24 | 5′-gga tgt gca gac gct gag ca-3′ |
| NR4A2 | SEQ ID NO: 25 | 5′-cct aag atg gaa atg acc tct c-3′ |
| | SEQ ID NO: 26 | 5′-ggg tga taa cac act cag cct-3′ |
| DSCR1L1 | SEQ ID NO: 27 | 5′-gct ctt agc gtt act ggt ctg-3′ |
| | SEQ ID NO: 28 | 5′-ggc aac ctc aga gtt ggg agt-3′ |
| A2M | SEQ ID NO: 29 | 5′-cct agc gca ttg cag gtg ct-3′ |
| | SEQ ID NO: 30 | 5′-cct ctg cct tgg tat atc cca-3t |
| SEPP1 | SEQ ID NO: 31 | 5′-cgt tgc tca gag gaa gca tct-3′ |
| | SEQ ID NO: 32 | 5′-cct agc cca tga att cig tct c-3′ |

The PCR product is cloned into Topo PCR2.1 vector (Invitrogen) to identify that the exact DNA site is cloned by DNA sequencing and then, the promoters of the cloned 8 kinds of genes were subjected to HpaII digestion assay, which is an analytical method capable of detecting promoter methylation.

After Caco2 (ATCC HTB 37) and HCT116 (ATCC CCL 247) cell lines derived from colon cancer tissue were cultured, genomic DNAs were extracted and each 200 ng of DNA was treated with 40 U of HpaII and 80 U of MspI, respectively. The DNAs treated with the restriction enzymes were purified, and then subjected to PCR amplification [(each consisting of 1 min at 94° C.→1 min at 66° C.→1 min at 72° C.) 30 cycles] using primers of Table 4 used in the above cloning, with DNA(mock DNA) which is not treated with a restriction enzyme.

The PCR products were electrophoresed to detect promoter methylation. Genes, whose promoters are methylated, have PCR product since they are not digested with HpaII and genes, whose promoters are not methylated, are not amplified since they are digested with HpaII. MspI restriction enzyme was used to detect that the activity of HpaII restriction enzyme was suppressed by methylated promoter. MspI digests by recognizing the same base sequence as the base sequence that HpaII restriction enzyme recognizes regardless of methylation, so that it is possible to detect the methylation-specific_digestion reaction of HpaII restriction enzyme.

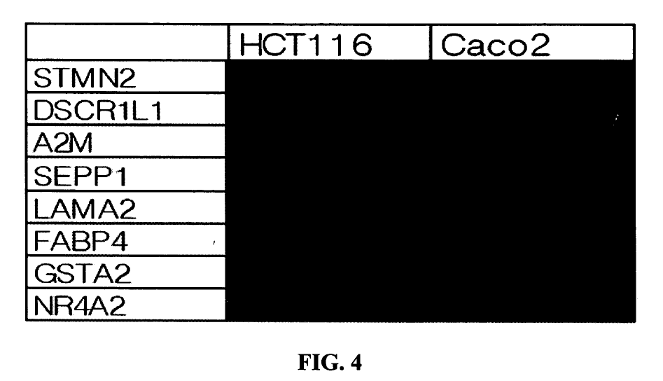

After electrophoresing the amplified PCR product, the case in which methylation is present, where PCR product is present in the samples treated with HpaII, is indicated by marking with a black block. The case in which PCR product is absent in the samples treated with HpaII is indicated by marking with a white block (FIG. 4). The results are shown in FIG. 4, and confirm that the promoters of the screened 8 kinds of genes are all methylated in colon cancer cell line.

### Example 4

### Bisulfite Sequencing

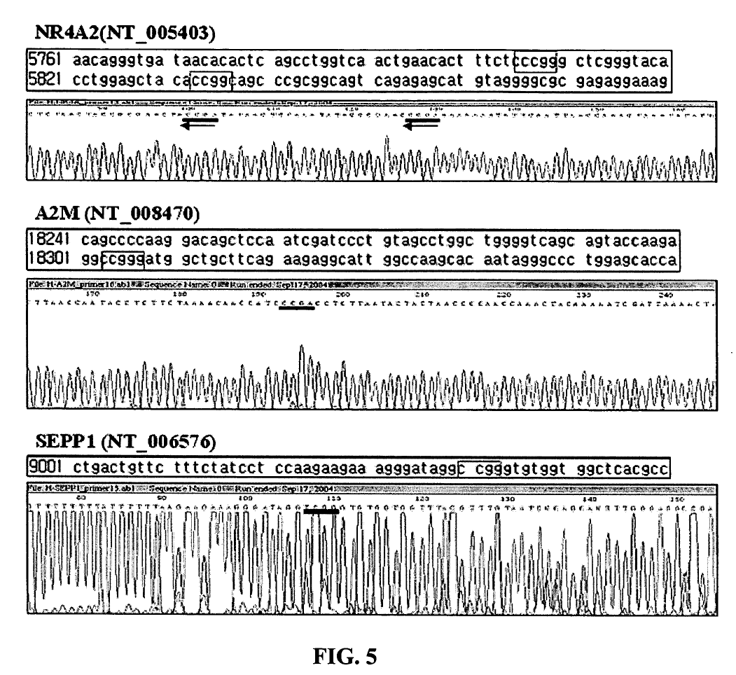

The promoters of 3 genes (NR4A2, A2M and SEPP1) among 8 kinds of genes was subjected to bisulfite sequencing to confirm whether the promoters of 8 kinds of genes, proved in Example 3, are methylated in another method (NR4A2: sequencing backwards; A2M and SEPP1: sequencing forwards). If DNAs are treated with bisulfite, non-methylated cytosine turns into uracil and methylated cytosine remains the same. 1 μg of genomic DNA of colon cancer cell line HCT116 was subjected to bisulfite modification (Sato, N. et al., Cancer Research, 63:3735, 2003) using a MSP bisulfite modification kit (In2Gen, Inc., Korea). Bisulfite treated HCT116 genomic DNA was subjected to PCR amplification using primers of SEQ ID NOs 25/26, SEQ ID NOs 29/30 and SEQ ID NOs 31/32, and then the sequences of PCR product were analyzed (FIG. 5). As a result, as shown in FIG. 5, cytosine was detected at all sites which includes CpG islands among sequences of the promoters of 3 genes (NR4A2, A2M and SEPP1), thereby providing confirmation that the promoters of the above 3 genes are all methylated.

### Example 5

### Clinical Assay Using the Promoters of 8 Genes

In order to confirm that the promoters of eight genes screened in the present invention can be applied to clinical practice, a methylation assay was performed on the clinical samples of colon cancer and lymphocyte. To fabricate a methylation microarray, promoter regions of SEQ ID NO: 9 to SEQ ID NO: 16 were obtained by PCR amplification and confirmed by sequencing. The obtained promoter regions were dissolved in 50% DMSO, spotted on a substrate using a GeneMachine OmniGrid spotter, air-dried for 24 hours, and then cross-linked by 3600 mJ UV, thereby fabricating the methylation microarray for cancer diagnosis.

Using the fabricated microarray, promoter methylation profiles were prepared on cancer tissues obtained from 17 colon cancer patients and lymphocyte samples obtained from 3 normal persons. For this purpose, genomic DNAs were first isolated from cancer tissues obtained from 17 colon cancer patients and lymphocyte samples obtained from 3 normal persons. The isolated DNAs were treated with HpaII restriction enzymes, respectively, and then subjected to PCR amplification [(each consisting of 1 min at 94° C.→1 min at 66° C.→1 min at 72° C.) 30 cycles] using primer mixtures of SEQ ID NO: 17 to SEQ ID NO: 32, thereby obtaining sample DNAs.

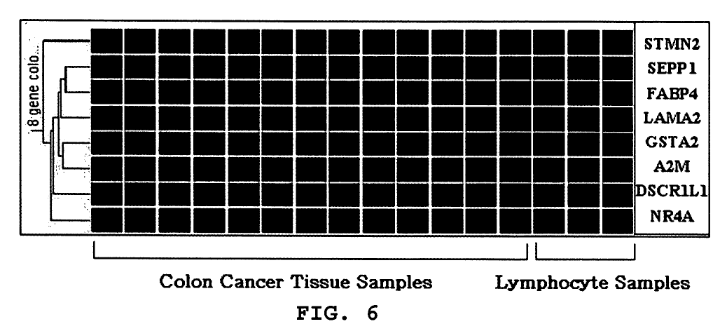

The amplified sample DNAs were hybridized with the fabricated methylation microarray for cancer diagnosis. Then, the case in which the hybridization in the HpaII-treated sample has been significantly increased was determined as reflecting methylation. This methylated case was indicated by marking with a red block. This shows a case where the PCR amplification in the HpaII-treated sample occurred significantly due to methylation. A case, where methylation did not occur, was indicated by marking with a black block (FIG. 6).

As shown in FIG. 6, it could be found that the promoter methylation of eight genes occurred in most of the colon cancer tissues, however, there was little or no methylation in lymphocyte samples known as normal control samples. Therefore it was confirmed that the promoters of 8 kinds of genes according to the present invention are useful in distinguishing effectively between normal tissues and cancer tissues by hierarchical clustering analysis.

As described above, in the present invention, eight cancer suppressor genes and their promoters were discovered, and it was revealed that they function as cancer diagnostic molecular markers that are widely applicable to various clinical samples.

The methylated promoters of the colon cancer-specific expression-decreased genes according to the present invention will be useful for the early detection of cancer. In addition, through the regulation of their expression, they will be useful as targets for screening new drugs allowing the early treatment of cancer. As a result, they are expected to contribute to the improvement of the complete cure rate of cancer.

While the present invention has been described with reference to particular illustrative embodiments, it is not intended to be restricted by such embodiments, but only by the appended claims. It is to be appreciated that those skilled in the art can change or modify such embodiments without departing from the scope and spirit of the present invention.

For example, the present invention contemplates nucleic acid sequences complementary to, or showing sequence similarity to, the DNA sequences specifically identified herein, as being potentially useful in the broad practice of the invention, including for example nucleotide sequences that are at least 60%, preferably at least 80%, and most preferably at least 95%, especially 98%, identical to specific sequences herein described. For example, polynucleotide sequences having at least 70% homology to polynucleotide sequences specifically disclosed herein, which retain the function of such specifically disclosed sequences for purposes of the invention, may find use in specific applications of the invention. The sequences that encode polypeptides may be identical to the sequences specifically disclosed herein or may be a different coding sequence, which coding sequence, as a result of the redundancy or degeneracy of the genetic code, encodes the same polypeptide as the polynucleotide sequences specifically disclosed herein. The polynucleotide sequences in accordance with the present invention may be in isolated form, e.g., as purified or removed from their original environment in any suitable manner.

Variants of polynucleotides may be naturally occurring allelic variants of the polynucleotides or non-naturally occurring variants of the polynucleotides. As is known in the art, an allelic variant is an alternate form of a polynucleotide sequence which may have a substitution, deletion or addition of one or more nucleotides, which does not substantially alter the function of the encoded polypeptide.

What is claimed is:

1. A methylated promoter of the colon cancer-specific expression-decreased gene selected from the group consisting of LAMA2, FABP4, GSTA2, STMN2, NR4A2, DSCR1L1, A2M and SEPP1.

2. The methylated promoter according to claim 1, wherein said LAMA2, FABP4, GSTA2, STMN2, NR4A2, DSCR1L1, A2M and SEPP1 has DNA sequence of SEQ ID NO: 1 to SEQ ID NO: 8 respectively.

3. The methylated promoter according to claim 1, wherein the promoter includes at least one methylated CpG dinucleotide.

4. The methylated promoter according to claim 1, wherein the promoter comprises any one DNA sequence selected from the group consisting of:
(a) 766-805 region of SEQ ID NO: 9;
(b) 896-935 region of SEQ ID NO: 9;
(c) 201-240 region of SEQ ID NO: 10;
(d) 206-245 region of SEQ ID NO: 11;
(e) 556-595 region of SEQ ID NO: 11;
(f) 661-700 region of SEQ ID NO: 11;
(g) 1176-1215 region of SEQ ID NO: 12;
(h) 81-120 region of SEQ ID NO: 13;
(i) 121-160 region of SEQ ID NO: 13;
(j) 226-265 region of SEQ ID NO: 14;
(k) 646-685 region of SEQ ID NO: 14;
(l) 226-265 region of SEQ ID NO: 15;
(m) 776-815 region of SEQ ID NO: 15;
(n) 266-305 region of SEQ ID NO: 16; and
(o) 476-515 region of SEQ ID NO: 16.

5. The methylated promoter according to claim 4, wherein the promoter has any one DNA sequence selected from the group consisting of SEQ ID NO: 9 to SEQ ID NO: 16.

6. A microarray for cancer detection, comprising a methylated promoter as claimed in claim 1, immobilized on a solid substrate.

7. The microarray for cancer detection according to claim 6, wherein the cancer is colon cancer.

8. A cancer diagnosis kit, comprising a methylated promoter as claimed in claim 1.

9. The cancer diagnosis kit according to claim 8, wherein the cancer is colon cancer.

10. A primer mixture containing at least one primer pair selected from the group consisting of SEQ ID NOs: 17/18, SEQ ID NOs: 19/20, SEQ ID NOs: 21/22, SEQ ID NOs: 23/24, SEQ ID NOs: 25/26, SEQ ID NOs: 27/28, SEQ ID NOs: 29/30, and SEQ ID NOs: 31/32.

11. A method for detecting promoter methylation of a clinical sample-derived gene, the method comprising the steps of:
(a) isolating sample DNAs from a clinical sample;
(b) treating the isolated sample DNAs with (i) an agent that modifies nonmethylated cytosine residues, or (ii) any one or more methylation sensitive restriction enzyme selected from the group consisting of HpaII, MspI, BssHII, BstUI and NotI;
(c) amplifying the treated DNAs using a primer capable of amplifying CpG island, which is originated from the promoter of the gene selected from the group consisting of LAMA2, FABP4, GSTA2, STMN2, NR4A2, DSCR1L1, A2M and SEPP1; and
(d) determining promoter methylation according to the presence of the amplified product in the step (c).

12. The method according to claim 11, wherein the clinical sample comprises tissue, cell, sputum, feces, urine, cell membrane, encephalon, amniotic fluid, eyeball, intestines, or blood derived from diagnosed subjects or cancer-suspected patients.

13. The method according to claim 11, wherein the agent that modifies nonmethylated cytosine residues comprises bisulfite.

14. The method according to claim 11, wherein the amplifying is carried out by PCR.

15. The method according to claim 11, wherein the primer capable of amplifying CpG island comprises a primer mixture containing at least one primer pair selected from the group consisting of SEQ ID NOs: 17/18, SEQ ID NOs: 19/20, SEQ ID NOs: 21/22, SEQ ID NOs: 23/24, SEQ ID NOs: 25/26, SEQ ID NOs: 27/28, SEQ ID NOs: 29/30, and SEQ ID NOs: 31/32.

16. The method according to claim 15, wherein the primer mixture contains all primers of SEQ ID NO: 17 to SEQ ID NO: 32.

17. The method according to claim 11, wherein step (d) comprises electrophoresing the amplified product obtained in step (c), and determining that a case, where the PCR product is present in the DNA treated with HpaII, is methylated, and a case, where the PCR product is absent in the DNA treated with HpaII, is nonmethylated, under the state where the PCR product is present in mock DNA.

18. The method according to claim 11, wherein step (d) comprises hybridizing the amplified product obtained in step (c) with a microarray for cancer detection comprising a methylated promoter immobilized on a solid substrate, wherein the methylated promoter comprises a colon cancer-specific expression-decreased gene selected from the group consisting of LAMA2, FABP4, GSTA2, STMN2, NR4A2, DSCR1L1, A2M and SEPP1, and determining that a case, where hybridization occurred in both mock DNA and the DNA treated with HpaII, is methylated.

19. A method for diagnosing cancer, the method comprises detecting methylation of CpG-containing promoter of the gene selected from the group consisting of LAMA2, FABP4, GSTA2, STMN2, NR4A2, DSCR1L1, A2M and SEPP1, in a clinical sample-derived DNA.

20. The method according to claim 19, wherein the detecting comprises the steps of:
(a) isolating sample DNAs from a clinical sample;
(b) treating the isolated sample DNAs with (i) an agent that modifies nonmethylated cytosine residues, or (ii) any one or more methylation sensitive restriction enzyme selected from the group consisting of HpaII, MspI, BssHII, BstUI and NotI;
(c) amplifying the treated DNAs using a primer capable of amplifying CpG island, which is originated from the promoter of the gene selected from the group consisting of LAMA2, FABP4, GSTA2, STMN2, NR4A2, DSCR1L1, A2M and SEPP1; and
(d) determining promoter methylation according to the presence of the amplified product in step (c).

21. The method according to claim 20, wherein the clinical sample is tissue, cell, sputum, feces, urine, cell membrane, encephalon, amniotic fluid, eyeball, intestines, or blood, derived from diagnosed subjects or cancer-suspected patients.

22. The method according to claim 20, wherein the agent that modifies nonmethylated cytosine residues comprises bisulfite.

23. The method according to claim 20, wherein the amplifying is carried out by PCR.

24. The method according to claim 20, wherein the primer capable of amplifying CpG island comprises a primer mixture containing at least one primer pair selected from the group consisting of SEQ ID NOs: 17/18, SEQ ID NOs: 19/20, SEQ ID NOs: 21/22, SEQ ID NOs: 23/24, SEQ ID NOs: 25/26, SEQ ID NOs: 27/28, SEQ ID NOs: 29/30, and SEQ ID NOs: 31/32.

25. The method according to claim 24, wherein the primer mixture contains all primers of SEQ ID NO: 17 to SEQ ID NO: 32.

26. The method according to claim 20, wherein step (d) comprises electrophoresing the amplified product obtained in step (c), and determining that a case, where the PCR product is present in the DNA treated with HpaII, is methylated, and a case, where the PCR product is absent in the DNA treated with HpaII, is nonmethylated, under the state where the PCR product is present in mock DNA.

27. The method according to claim 20, wherein step (d) comprises hybridizing the amplified product in the step (c) with a microarray for cancer detection comprising a methylated promoter immobilized on a solid substrate, wherein the methylated promoter comprises a colon cancer-specific expression-decreased gene selected from the group consisting of LAMA2, FABP4, GSTA2, STMN2, NR4A2, DSCR1L1, A2M and SEPP1, and determining that a case, where hybridization occurred in both the mock DNA and the DNA treated with HpaII, is methylated.

28. The method according to claim 20, wherein the cancer is colon cancer.

29. A polynucleotide, comprising a methylated promotor of a nucleotide sequence selected from the group consisting of LAMA2, FABP4, GSTA2, STMN2, NR4A2, DSCR1L1, A2M and SEPP1a nucleotide sequences, wherein said polynucleotide includes at least one methylated CpG dinucleotide.

30. The polynucleotide of claim 29, wherein said promoter includes a promoter sequence selected from the group consisting of:
(a) 766-805 region of SEQ ID NO: 9;
(b) 896-935 region of SEQ ID NO: 9;
(c) 201-240 region of SEQ ID NO: 10;
(d) 206-245 region of SEQ ID NO: 11;
(e) 556-595 region of SEQ ID NO: 11;
(f) 661-700 region of SEQ ID NO: 11;
(g) 1176-1215 region of SEQ ID NO: 12;
(h) 81-120 region of SEQ ID NO: 13;
(i) 121-160 region of SEQ ID NO: 13;
(j) 226-265 region of SEQ ID NO: 14;
(k) 646-685 region of SEQ ID NO: 14;
(l) 226-265 region of SEQ ID NO: 15;
(m) 776-815 region of SEQ ID NO: 15;
(n) 266-305 region of SEQ ID NO: 16; and
(o) 476-515 region of SEQ ID NO: 16.
